(19) United States
(12) Patent Application Publication
(10) Pub. No.: US 2023/0347015 A1
(43) Pub. Date: Nov. 2, 2023

(54) LYOPHILIZED PERFORATED PLACENTAL MEMBRANE TISSUE GRAFTS

(71) Applicant: MiMedx Group, Inc., Marietta, GA (US)

(72) Inventors: Thomas J. Koob, Santa Fe, NM (US); Michelle Massee, Milton, GA (US); Divya Sukumar, Brookhaven, GA (US)

(21) Appl. No.: 18/296,207

(22) Filed: Apr. 5, 2023

Related U.S. Application Data
(60) Provisional application No. 63/362,540, filed on Apr. 6, 2022.

(51) Int. Cl. A61L 27/36

(52) U.S. Cl. CPC A61L 27/3604

(57) ABSTRACT

A dehydrated, partially perforated, placental tissue allograft that can be used in the treatment of wounds. Specifically, the dehydrated, partially perforated placental allograft has the property of being able to be cleaned during processing without disrupting the grafts structural integrity or decreasing the concentration of growth factors found within the finished graft. The dehydrated, partially perforated, placental tissue allograft is reconstituted prior to applying it to the subject and is substantially free of undesirable maternal blood contaminants.

### CROSS REFERENCE TO RELATED APPLICATION

This application claims priority from U.S. provisional application Ser. No. 63/362,540, filed Apr. 6, 2022, which is hereby incorporated by reference in its entirety for all of its teachings.

### FIELD OF THE INVENTION

The invention relates to lyophilized, perforated placental membrane tissue grafts for use in the treatment of various chronic wounds.

### BACKGROUND

Human placental membrane (e.g. amniotic membrane or tissue) has been used for various types of reconstructive surgical procedures since the early 1900s. The membrane serves as a substrate material, more commonly referred to as a biological dressing or patch graft. Such membrane has also been used widely for ophthalmic procedures in the United States and in countries in the southern hemisphere. Typically, such membrane is either frozen or dried for preservation and storage until needed for surgery.

Such placental tissue is typically harvested after an elective Cesarean surgery or natural delivery. The placenta has three primary layers of tissue including amniotic membrane, the intermediate layer (or spongy layer), and chorion. The amniotic membrane is a non-vascular tissue that is the innermost layer of the placenta, and consists of a single layer, which is attached to a basement membrane. Histological evaluation indicates that the membrane layers of the amniotic membrane consist of epithelium cells, thin reticular fibers (basement membrane), a thick compact layer, and fibroblast layer. The fibrous layer of amnion (i.e., the basement membrane) contains cell anchoring collagen types IV, V, and VII. The chorion is also considered as part of the fetal membrane; however, the amniotic layer and chorion layer are separate and separable entities. The chorion comprises a reticular layer, a basement membrane, and a trophoblast layer. The intermediate layer is found in between the amniotic membrane and the chorion, loosely connecting the two, adjacent to the amnion's fibroblast layer and the chorion's reticular layer.

Amniotic membrane and chorion tissue provide unique grafting characteristics when used for surgical procedures, including providing a matrix for cellular migration/proliferation, providing a natural biological barrier, are non-immunogenic, promote increased self-healing, are susceptible of being fixed in place using different techniques including fibrin glue or suturing. And, such grafts, when properly prepared, can be stored at room temperature for extended periods of time, without need for refrigeration or freezing, until needed for a surgical procedure.

Known clinical procedures or applications for such placental membrane grafts include Schneiderian Membrane repair (i.e. sinus lift), guided tissue regeneration (GTR), general wound care, and primary closure membrane. Known clinical procedures or applications for such placental membrane grafts include biological would dressing.

A detailed look at the history and procedure for harvesting and using “live” amniotic tissue for surgical procedures and a method for harvesting and freezing amniotic tissue grafts for ophthalmic procedures is described in U.S. Pat. No. 6,152,142 issued to Tseng, which is incorporated herein by reference in its entirety.

However, placental membrane may present challenges during processing, such as the removal of residual maternal blood from the intermediate layer of the placental membrane, which may potentially result in immune reactions in the patient. Additionally, the presence of blood specks and clots can result in an end product that is not aesthetically pleasing to end users, such as patients or healthcare practitioners. Accordingly, manufacturers have developed a variety of techniques to clean the tissue to reduce the chance of an immune reaction and to create a more aesthetically pleasing product that will appeal to patients who receive placental tissue grafts as well as the healthcare practitioners who apply them in the field.

### SUMMARY OF THE INVENTION

An embodiment of the invention is a placental tissue graft comprising a layer of amnion, intermediate layer, and a layer of chorion;
wherein said intermediate layer has been fully or partially perforated; and
wherein said amnion has not been perforated.

An embodiment of the invention is a placental tissue graft comprising a layer of amnion, an intermediate layer, and a layer of chorion;
wherein the intermediate layer comprises a first region and a second region, wherein the first region is adjacent to the amnion and the second region is adjacent to the chorion;
wherein the second region has been fully perforated; and
wherein the first region has not been perforated.

An embodiment of the invention is a method of preparing a placental tissue graft comprising amnion, intermediate layer, and chorion, said method comprising the steps of:
a. separating an amnion layer from a chorion layer, so as to obtain an amnion layer with some intermediate layer attached and a chorion layer with some intermediate layer attached;
b. perforating the intermediate layer that is attached to the chorion layer;
c. washing the amnion layer and the chorion layer; and
d. contacting the intermediate layer attached to the amnion layer with the intermediate layer attached to the chorion layer,

thereby producing a placental tissue graft comprising amnion, intermediate layer, and chorion.

In certain embodiments, the subject matter described herein is directed toward a dehydrated, selectively perforated placental membrane tissue graft, and particularly human allograft, that is processed to preserve the spongy intermediate layer, while also removing undesirable blood remnants and clots. Additionally, the perforated placental membrane tissue graft is dehydrated to enhance its shelf life and durability.

After harvesting, the placental membrane tissue is treated in a number of steps to provide the products described herein. For example, amniotic membrane and the chorion layers are separated from other placental tissue, such as the umbilical cord and the placental disc tissue (the other tissues may be retained for other purposes). All components are sourced from a single donor. Once the amnion and chorion membrane has been separated from the other tissue components, the amnion layer is physically pulled apart from the chorion layer, resulting in isolated amnion and chorion layers. The separated layers are then washed in sterile water.

After the layers of amnion and chorion have been rinsed in the sterile water, they are placed on a flat surface. The chorion layer is situated with the spongy intermediate layer facing up. Blood, blood clots and other debris should be removed from the amnion and chorion layers by using gauze, forceps, and/or gloved hands. Once the tissue layers have been properly situated, the intermediate layer attached to the chorion layer is subjected to full or partial thickness perforation using a specialized rotary pinwheel tool.

The amniotic tissue is gently cleansed and minimally manipulated to preserve inherent growth factors and proteins in the tissue. Notable growth factors in the amniotic tissue include transforming growth factor beta (TGF-β), basic fibroblast growth factor (bFGF), platelet derived growth factors (PDGF AA & BB), and vascular endothelial growth factor (VEGF)14,15, which are known to regulate wound healing.

Once the cleaning and perforation steps have been completed, the amnion and chorion layers, with attached intermediate layer, should be subjected to an additional rinsing in sterile water. After the sterile water rinse is complete, the amnion and chorion layers are subjected to a decontamination step, where they are rinsed in a solution containing one or more broad spectrum antibiotics, such as Streptomycin Sulfate, Gentamicin Sulfate, Polymixin B Sulfate and/or Bacitracin

After the rinsing and decontamination steps have been carried out, the selected layers of placental tissue are reassembled and are then subjected to a drying process, which may involve any type of commercially acceptable process known in the art, including, but not limited to air drying, chemical drying or lyophilization of the amnion and chorion layers.

In one embodiment, the selected tissue layers are dried on a fixture. The surface of the drying fixture has a plurality of grooves that defines the outer contours of each of the plurality of placenta membrane tissue grafts and wherein the step of cutting comprises cutting the selected layer along the grooves.

The finished product is packaged in a sterile container and is reconstituted with an acceptable excipient by the end user before the graft is applied to the subject's wound site. Alternatively, the graft can be applied directly to the wound site and can be reconstituted with a combination of an excipient and the patient's own bodily fluids that may be present in the wound site.

### BRIEF DESCRIPTION OF THE FIGURES

Further features and benefits of the present invention will be apparent from a detailed description of preferred embodiments thereof taken in conjunction with the following drawings, wherein similar elements are referred to with similar reference numbers, and wherein:

FIG. 1 is a photograph of an exemplary specialized rotary pinwheel micro needling tool.

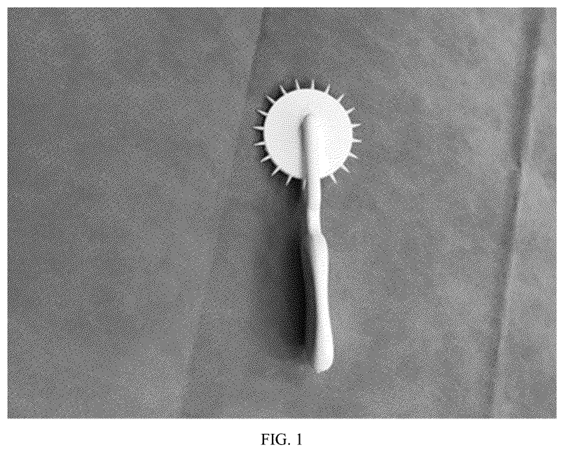

FIG. 2 is a schematic diagram of a graft according to the invention.

### DETAILED DESCRIPTION OF THE INVENTION

One challenge that has been encountered in the field is that placental tissue grafts can retain blood, blood clots and other undesirable contaminants that can negatively impact the appearance of the finished product, and may also provoke an unwanted immune response in the patient to whom the graft has been applied. Manufacturers have developed a number of ways to help remove these blood contaminants during processing.

Accordingly, there is a need in the marketplace for an efficient manner of removing as much blood, blood clots and other undesirable contaminants as possible, particularly from the intermediate layer, without damaging the structural integrity of the tissue itself or compromising the concentration of growth factors that remain in the tissue grafts after the tissues have been subjected to the manufacturing process.

It is to be understood that this invention is not limited to particular embodiments described, as such may, of course, vary. It is also to be understood that the terminology used herein is for the purpose of describing particular embodiments only, and is not intended to be limiting, since the scope of this invention will be limited only by the appended claims.

The detailed description of the invention is divided into various sections only for the reader's convenience and disclosure found in any section may be combined with that in another section. Unless defined otherwise, all technical and scientific terms used herein have the same meaning as commonly understood by one of ordinary skill in the art to which this invention belongs. Although any methods and materials similar or equivalent to those described herein can also be used in the manufacture, practice or testing of the present invention, the preferred methods and materials are now described. All patents and publications mentioned herein are incorporated by reference to disclose and describe the methods and/or materials in connection with which the publications are cited.

Each embodiment disclosed herein is contemplated as being applicable to each of the other disclosed embodiments. All combinations and sub-combinations of the various elements described herein are within the scope of the embodiments.

It is understood that where a parameter range is provided, all integers and ranges within that range, and tenths and hundredths thereof, are also provided by the embodiments. For example, “5-10%” includes 5%, 6%, 7%, 8%, 9%, and 10%; 5.0%, 5.1%, 5.2% . . . 9.8%, 9.9%, and 10.0%; and 5.00%, 5.01%, 5.02% . . . 9.98%, 9.99%, and 10.00%, as well as, for example, 6-9%, 5.1%-9.9%, and 5.01%-9.99%. This also applies to ratios. For example, a recited ratio range of “1:100 to 200:1” includes ratios such as 1:50, 1:1, and 100:1, along with ranges such as 1:100 to 1:1, 1:50 to 50:1, and 1:1 to 200:1.

As used herein, “about” in the context of a numerical value or range means within ±1%, ±5%, or 10% of the numerical value or range recited or claimed.

The invention illustratively disclosed herein suitably may be practiced in the absence of any element which is not specifically disclosed herein.

It must be noted that as used herein and in the appended claims, the singular forms “a”, “an”, and “the” include plural referents unless the context clearly dictates otherwise.

### Definitions

As used herein the following terms have the following meanings.

“Placental tissue” or “placenta” refers to placental amnion, placental intermediate layer, and placental chorion.

“Comprising” or “comprises” is intended to mean that the compositions, for example media, and methods include the recited elements, but not excluding others. “Consisting essentially of” when used to define methods, shall mean excluding other elements of any essential significance to the combination for the stated purpose. “Consisting of” shall mean excluding additional substantial method steps. Embodiments defined by each of these transition terms are within the scope of this invention.

“Dehydrated” means that the tissue has had substantially all of its water removed, (i.e. greater than 85%, greater than 90%, greater than 95%, greater than 99%, or 100% of its water removed).

“Substantially uniform,” with respect to the intermediate layer thickness, means that the thickness is ±20%, ±15%, ±10%, ±5%, or ±1% throughout the entirety of the graft

“Optional” or “optionally” means that the subsequently described event or circumstance can or cannot occur, and that the description includes instances where the event or circumstance occurs and instances where it does not.

The term “subject” as used herein is any vertebrate organism including but not limited to mammalian subjects such as humans, farm animals, domesticated pets and the like. The term “patient” may be used interchangeably with “subject.”

The term “treat,” with respect to a wound, means to reduce the amount of time the wound would have taken to heal in the absence of any type of medical intervention.

A tissue layer that has been “fully perforated” has had at least one microneedle penetrate the entire thickness of the layer. A tissue layer that has been “partially perforated” has had at least one microneedle penetrate part of the entire thickness of the layer. A tissue layer that has been fully or partially perforated may or may not retain perforations following further processing of the tissue layer.

The term “microneedle,” as used herein, refers to any device capable of fully or partially penetrating a tissue without removing any surface area of the tissue. Non-limiting examples of microneedles include needles and pins.

An embodiment of the invention is a placental tissue graft comprising a layer of amnion, intermediate layer, and a layer of chorion;
wherein said intermediate layer has been fully or partially perforated; and
wherein said amnion has not been perforated.

In an embodiment, the intermediate layer has been fully perforated. In an embodiment, the intermediate layer has been partially perforated.

An embodiment of the invention is a placental tissue graft comprising a layer of amnion, an intermediate layer, and a layer of chorion;
wherein the intermediate layer comprises a first region and a second region, wherein the first region is adjacent to the amnion and the second region is adjacent to the chorion;
wherein the second region has been fully perforated; and
wherein the first region has not been perforated.

In an embodiment, the chorion has been partially perforated. In an embodiment, the chorion has not been partially perforated. In an embodiment, the chorion has not been perforated.

In an embodiment, the intermediate layer is substantially uniform throughout the graft.

In an embodiment, the placental tissue allograft is dehydrated. In an embodiment, the placental tissue allograft is lyophilized.

In an embodiment, the placental tissue allograft is contained within a sealed pouch. In an embodiment, the sealed pouch is deoxygenated.

An embodiment of the invention is a method of preparing a placental tissue graft comprising amnion, intermediate layer, and chorion, said method comprising the steps of:
a. separating an amnion layer from a chorion layer, so as to obtain an amnion layer with some intermediate layer attached and a chorion layer with some intermediate layer attached;
b. perforating the intermediate layer that is attached to the chorion layer;
c. washing the amnion layer and the chorion layer; and
d. contacting the intermediate layer attached to the amnion layer with the intermediate layer attached to the chorion layer,

thereby producing a placental tissue graft comprising amnion, intermediate layer, and chorion.

In an embodiment, the method further comprises dehydrating the placental tissue graft following step d. In an embodiment, the dehydrating comprises lyophilization.

In an embodiment, the amnion is not perforated during the method.

In an embodiment, the method further comprises a step of manipulating the intermediate layer attached to the amnion and/or chorion so as to make the intermediate layer more uniformly distributed. In an embodiment, making the intermediate layer more uniformly distributed results in the intermediate layer substantially uniformly distributed throughout the entirety of the graft.

In an embodiment, steps b. and c. are each performed more than once.

In an embodiment, the method further comprises disrupting the amnion's epithelial layer. In an embodiment, the method further comprises substantially removing the amnion's epithelial layer.

An embodiment is a graft prepared by any of these methods.

An embodiment of the invention is also a method of treating or covering a wound, comprising contacting a wound with a graft as described herein.

### Methods of Manufacture

### Initial Tissue Collection

The recovery of placenta tissue originates in a hospital, where it is collected during a Cesarean section birth or natural birth. The donor, referring to the mother who is about to give birth, voluntarily submits to a comprehensive screening process designed to provide the safest tissue possible for transplantation. The screening process preferably tests for antibodies to the human immunodeficiency virus type 1 and type 2 (anti-HIV-1 and anti-HIV-2), hepatitis B surface antigens (HBsAg), antibodies to the hepatitis C virus (anti-HCV), antibodies to the human T-lymphotropic virus type I and type II (anti-HTLV-I and anti-HTLV-II), CMV, and syphilis, using conventional serological tests. The above list of tests is exemplary only, as more, fewer, or different tests may be desired or necessary over time or based upon the intended use of the grafts, as will be appreciated by those skilled in the art.

Based upon a review of the donor's information and screening test results, the donor will either be deemed acceptable or not. In addition, at the time of delivery, cultures are taken to determine the presence of, for example, Clostridium or Streptococcus. If the donor's information, screening tests, and the delivery cultures are all negative (i.e., do not indicate any risks or indicate acceptable level of risk), the donor is approved and the tissue specimen is designated as initially eligible for further processing and evaluation.

Human placentas that meet the above selection criteria are preferably individually bagged in a saline solution in a sterile shipment bag and stored in a container of wet ice for shipment to a processing location or laboratory for further processing.

### Material Check-In and Evaluation

Upon arrival at the processing center or laboratory, the shipment is opened and verified that the sterile shipment bag/container is still sealed and intact, that ice or other coolant is present and that the contents are cool, that the appropriate donor paperwork is present, and that the donor number on the paperwork matches the number on the sterile shipment bag containing the tissue. The sterile shipment bag containing the tissue is then stored in a refrigerator until ready for further processing. All appropriate forms are completed and chain of custody and handling logs are also completed.

### Gross Tissue Processing Step

When the tissue is ready to be processed further, the sterile supplies necessary for processing the placenta tissue further are assembled in a staging area in a controlled environment and are prepared for introduction into a critical environment. If the critical environment is a manufacturing hood, the sterile supplies are opened and placed into the hood using conventional sterile technique. If the critical environment is a clean room, the sterile supplies are opened and placed on a cart covered by a sterile drape. All the work surfaces are covered by a piece of sterile drape using conventional sterile techniques, and the sterile supplies and the processing equipment are placed on to the sterile drape, again using conventional sterile technique.

If the placental tissue is collected prior to the completion or obtaining of results from the screening tests and delivery cultures, such tissue is labeled and kept in quarantine. The tissue is approved for further processing only after the required screening assessments and delivery cultures, which declare the tissue safe for handling and use, are satisfied.

Processing equipment is decontaminated according to conventional and industry-approved decontamination procedures and then introduced into the critical environment. The equipment is strategically placed within the critical environment to minimize the chance for the equipment to come in proximity to or be inadvertently contaminated by the tissue specimen.

Next, the placenta is removed from the sterile shipment bag and transferred aseptically to a sterile processing basin within the critical environment. The sterile basin contains, preferably, sterile water that is at room or near room temperature. The placenta is gently massaged to help separate blood clots and to allow the placenta tissue to reach room temperature, which will make the separation of the amnion and chorion layers from each other, as discussed hereinafter, easier. After having warmed up to the ambient temperature (after about 10-30 minutes), the placenta is then removed from the sterile processing basin and laid flat on a processing tray with the amniotic membrane layer facing down for inspection.

The placenta tissue is examined and the results of the examination are documented on a “Raw Tissue Assessment Form.” The placenta tissue is examined for discoloration, debris or other contamination, odor, and signs of damage. The size of the tissue is also noted. A determination is made, at this point, as to whether the tissue is acceptable for further processing.

Next, if the placenta tissue is deemed acceptable for further processing, the amnion and chorion layers of the placenta tissue are then carefully separated. The materials and equipment used in this procedure include the processing tray, sterile water, sterile 4×4 inch sponges, and two sterile Nalgene jars. The placenta tissue is then closely examined to find an area (typically a corner) in which the amniotic membrane layer can be separated from the chorion layer. The amniotic membrane appears as a thin, opaque layer on the chorion.

With the placenta tissue in the processing tray with the amniotic membrane layer facing down, the chorion layer is gently lifted off the amniotic membrane layer in a slow, continuous motion, using care to prevent tearing of the amniotic membrane. If a tear starts, it is generally advisable to restart the separation process from a different location to minimize tearing of either layer of tissue. The separation process continues by hand, without the use of the sponges, being careful not to tear either the amnion layer or the chorion layer. Following separation, the majority of the intermediate layer will be on the chorion, and the remainder of the intermediate layer will be on the amnion.

Care is then taken to remove blood clots and other extraneous tissue from each layer of tissue until the amniotic membrane tissue and the chorion are clean and ready for further processing. More specifically, the amnion and chorion tissues are placed on the processing tray and blood clots are carefully removed using a blunt instrument, a finger, or a sterile non-particulating gauze, by gently rubbing the blood until it is free from the stromal tissue of the amnion and from the trophoblast tissue of the chorion. The stromal layer of the amnion is the side of the amniotic membrane that faces the mother. In contrast, the basement membrane layer is the side of the amnion that faces the fetus.

Using a blunt instrument, such as a forceps, or sterile gauze, any residual debris or contamination is also removed. This step must be done with adequate care, again, so as not to tear the amnion or chorion tissues, and so as not to disrupt or remove the intermediate layer from the amnion or chorion. The cleaning of the amnion is complete once the amnion tissue is smooth and opaque-white in appearance with a clear intermediate layer. If the chorion tissue is cleaned too much, the opaque layer can be removed. Any areas of the chorion cleaned too aggressively and appearing clear will be unacceptable and will ultimately be discarded.

### Microneedling of the Chorion Layer Step

Once the amnion and chorion layers have been separated and cleaned, a microneedling tool is used to perforate the intermediate layer which is attached to the chorion layer. In an embodiment, the microneedling tool is applied at a 45 degree angle from the surface of the chorion approximately 10-15 times, approximately 2 cm apart. In an embodiment, the chorion layer is then rotated 90 degrees from its original orientation, and the tool is again applied 10-15 times, approximately 2 cm apart. It is important that the microneedling tool be applied with enough pressure to penetrate the intermediate layer, but should not penetrate completely through the entire layer of chorion. In some embodiments, the microneedling tool does not penetrate the chorion's basement layer.

### Optional Epithelial Cell Removal Step

In some embodiments, the epithelium layer present on the amnion is substantially removed in order to expose the basement layer of the amnion. The term “substantially removed” with respect to the amount of epithelium removed is defined herein as removing greater than 90%, greater than 95%, or greater than 99% of the epithelial cells from the amnion. In some embodiments, the majority (greater than 50%) of the epithelial cells are removed. In some embodiments, greater than 55%, greater than 60%, greater than 65%, greater than 70%, greater than 75%, greater than 80%, or greater than 85%, of the epithelial cells are removed. The presence or absence of epithelial cells remaining on the amnion layer can be evaluated using techniques known in the art. For example, after removal of the epithelial cell layer, a representative tissue sample from the processing lot is placed onto a standard microscope examination slide. The tissue sample is then stained using Eosin Y Stain and evaluated as described below. The sample is then covered and allowed to stand. Once an adequate amount of time has passed to allow for staining, visual observation is done under magnification.

The epithelium layer can be removed by techniques known in the art, in some embodiments. For example, the epithelium layer can be scraped off of the amnion using a cell scraper. Other techniques include, but are not limited to, freezing the membrane, physical removal using a cell scraper, or exposing the epithelial cells to nonionic detergents, anionic detergents, and nucleases. The de-epithelialized tissue is then evaluated to determine that the basement membrane has not been compromised and remains intact. This step is performed after completion of the processing step and before the tissue has been dehydrated. For example, a representative sample graft is removed for microscopic analysis. The tissue sample is place onto a standard slide, stained with Eosin Y and viewed under the microscope. If epithelium is present, it will appear as cobblestone-shaped cells. In embodiments where the epithelial layer is removed, it is understood that it will be removed in a way which does not result in removal of the intermediate layer.

### Chemical Decontamination Step

Each membrane tissue is then placed into a sterile Nalgene jar for the next step of chemical decontamination.

Next, each Nalgene jar is aseptically filled with sterile water and sealed (or closed with a top). The jar is then placed on a rocker platform and agitated for between 30 and 90 minutes, which further cleans the tissue of contaminants.

If the rocker platform was not in the critical environment (e.g., the manufacturing hood), the Nalgene jar is returned to the critical/sterile environment and opened. Using sterile forceps, the tissue is gently removed from the Nalgene jar containing sterile water and placed into an empty Nalgene jar. This empty Nalgene jar with the tissue is then aseptically filled with a pre-mixed antibiotic solution. Preferably, the premixed antibiotic solution is comprised of a cocktail of antibiotics, such as Streptomycin Sulfate and Gentamicin Sulfate. Other antibiotics, such as Polymixin B Sulfate and Bacitracin, or similar antibiotics now available or available in the future, are also suitable. Additionally, it is preferred that the antibiotic solution be at room temperature when added so that it does not change the temperature of or otherwise damage the tissue. This jar or container containing the tissue and antibiotics is then sealed or closed and placed on a rocker platform and agitated for, preferably, between 60 and 90 minutes. Such rocking or agitation of the tissue within the antibiotic solution further cleans the tissue of contaminants and bacteria.

Again, if the rocker platform was not in the critical environment (e.g., the manufacturing hood), the jar or container containing the tissue and antibiotics is then returned to the critical/sterile environment and opened. Using sterile forceps, the tissue is gently removed from the jar or container and placed in a sterile basin containing sterile water or normal saline (0.9% saline solution). The tissue is allowed to soak in place in the sterile water/normal saline solution for at least 10 to 15 minutes. The tissue may be slightly agitated to facilitate removal of the antibiotic solution and any other contaminants from the tissue. After at least 10 to 15 minutes, the tissue is ready to be dehydrated and processed further. Alternatively, the microneedling and/or chemical decontamination steps may be repeated at this time, to remove additional contaminants.

### Optional Disruption of Epithelial Layer Step

In embodiments where the epithelial layer is present, the amnion's epithelial layer may be disrupted. In embodiments, this comprises lightly scraping the epithelial layer with a sterile device, such as a ruler.

### Recombination Step

In an embodiment, the amnion and chorion are recombined prior to dehydration. The stromal side of the amnion (with some intermediate layer attached) is placed against the reticular layer side of the chorion (with some intermediate layer attached). In an embodiment, the intermediate layer on one or both of the amnion and chorion is manipulated so as to be more uniform, and/or to move intermediate layer to portions of the graft which have little or no intermediate layer. This manipulation may be performed with any suitable sterile tool, including but not limited to a ruler. In an embodiment, this manipulation is performed prior to recombination of the amnion and the chorion. In an embodiment, this manipulation is performed after recombination of the amnion and the chorion.

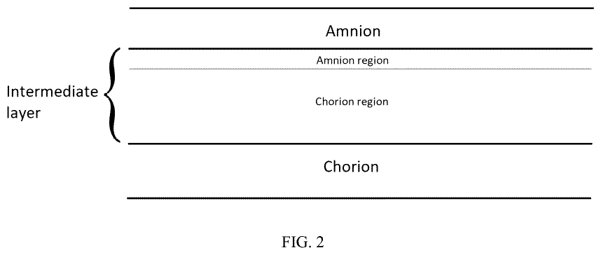

The graft following recombination is depicted in schematic form in FIG. 2 (not to scale). The intermediate layer is found between the amnion and the chorion. There are two regions of the intermediate layer: a first region, adjacent to the amnion (“amnion region”), and a second region, adjacent to the chorion (“chorion region”). The first region will not have been perforated during processing and forming of the graft. The second region will have been fully perforated during processing and forming of the graft.

### Dehydration Step

Preferably, the placental tissue is placed in an individual, sealed Tyvek pouch (or other commercially available pouch) and placed into a commercially available freeze drying chamber. Any lyophilization process known to one skilled in the art may be used, so long as the placental tissue is substantially dehydrated when the lyophilization process has been completed.

Other methods may be used to adequately dehydrate the placental tissue. Such techniques may include, but are not limited to chemical dehydration, or placing the placental tissue in a low humidity/high temperature environment for an adequate period of time until optimal dehydration of the placental tissue has been achieved. Such dehydration techniques are generally well-known to those having skill in the art.

### Sterilization

The inner and outer pouch along with the resulting dehydrated placental tissue grafts are subjected to a terminal sterilization step. Terminal sterilization is accomplished by exposing the dehydrated placental tissue grafts to high energy, penetrating, ionizing radiation such as electron beam or gamma irradiation while the product is in its final packaging unit.

### Reconstitution of Dehydrated, Perforated Placental Tissue Graft

In order to administer the placental tissue graft to a subject, the end user may first reconstitute the graft by rehydrating it. Optimally, the rehydrating agent is 0.9% saline solution, but any suitable excipient may be used.

### Administration of Perforated Placental Tissue Graft

Once the placental tissue graft has been reconstituted with the desired rehydrating agent, it is then applied to the wound site. Also, the graft may be hydrated in the wound site with the rehydrating agent or blood present from wound bed preparation. The reconstituted placental tissue graft may also have patterns applied to it that indicate whether the amnion or chorion layer is facing the wound site.

Efforts have been made to ensure accuracy with respect to numbers used (e.g. amounts, temperature, etc.) but some experimental errors and deviations should be accounted for.

Unless defined otherwise, technical and scientific terms used herein have the same meaning as commonly understood by one of ordinary skill in the art to which this subject matter belongs, and are consistent with: Singleton et al (1994) Dictionary of Microbiology and Molecular Biology, 2nd Ed., J. Wiley & Sons, New York, NY; and Janeway, C., Travers, P., Walport, M., Shlomchik (2001) Immunobiology, 5th Ed., Garland Publishing, New York.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation. One skilled in the art will recognize many methods and materials similar or equivalent to those described herein, which could be used in the practicing the subject matter described herein. The present disclosure is in no way limited to just the methods and materials described.

1. A placental tissue graft comprising a layer of amnion, intermediate layer, and a layer of chorion;
wherein said intermediate layer has been fully or partially perforated; and
wherein said amnion has not been perforated.

2. The placental tissue graft of claim 1, wherein said intermediate layer has been fully perforated.

3. The placental tissue graft of claim 1, wherein said intermediate layer has been partially perforated.

4. A placental tissue graft comprising a layer of amnion, an intermediate layer, and a layer of chorion;
wherein the intermediate layer comprises a first region and a second region, wherein the first region is adjacent to the amnion and the second region is adjacent to the chorion;
wherein the second region has been fully perforated;
wherein the first region has not been perforated; and
wherein the amnion has not been perforated.

5. The placental tissue graft of claim 1, wherein said chorion has been partially perforated.

6. The placental tissue graft of claim 1, wherein said chorion has not been partially perforated.

7. The placental tissue graft of claim 1, wherein said intermediate layer is substantially uniform throughout the graft.

8. The placental tissue graft of claim 1, wherein said placental tissue graft is dehydrated.

9. The placental tissue graft of claim 1, wherein said placental tissue graft is rehydrated.

10. The placental tissue graft of claim 1, wherein said graft is contained in a sealed pouch.

11. The placental tissue graft of claim 10, wherein said sealed pouch is deoxygenated.

12. A method of preparing a placental tissue graft comprising amnion, intermediate layer, and chorion, said method comprising the steps of:
a) separating an amnion layer from a chorion layer, so as to obtain an amnion layer with some intermediate layer attached and a chorion layer with some intermediate layer attached;
b) perforating the intermediate layer that is attached to the chorion layer;
c) washing the amnion layer and the chorion layer; and
d) contacting the intermediate layer attached to the amnion layer with the intermediate layer attached to the chorion layer,
thereby producing a placental tissue graft comprising amnion, intermediate layer, and chorion.

13. The method of claim 12, further comprising dehydrating the placental tissue graft following step d).

14. (canceled)

15. The method of claim 12, wherein said amnion is not perforated during the method.

16. The method of claim 12, further comprising a step of manipulating the intermediate layer attached to the amnion and/or chorion so as to make the intermediate layer more uniformly distributed.

17. The method of claim 12, wherein steps b) and c) are each performed more than once.

18. The method of claim 12, further comprising disrupting the amnion's epithelial layer.

19. The method of claim 12, further comprising substantially removing the amnion's epithelial layer.

20. A graft prepared by the method of claim 12.

21. A method of treating a wound, said method comprising contacting the wound with a graft of claim 1.
